(19) United States
(12) Patent Application Publication
(10) Pub. No.: US 2025/0134591 A1
(43) Pub. Date: May 1, 2025

(54) MEDICAL DEVICE AND METHODS OF USE

(71) Applicant: BOSTON SCIENTIFIC SCIMED, INC., Maple Grove, MN (US)

(72) Inventors: Enda Connaughton, Galway (IE); Richard Crawford, Athenry (IE); Aiden Flanagan, Kilcolgan (IE); Niamh Fitzgerald, Oranmore (IE); Michael Hughes, Claregalway (IE); Anthony O'Brien, Oranmore (IE); Niraj Prasad Rauniyar, Plymouth, MN (US); Anthony Frank Tassoni, JR., Andover, MN (US)

(73) Assignee: BOSTON SCIENTIFIC SCIMED, INC., Maple Grove, MN (US)

(21) Appl. No.: 18/926,064

(22) Filed: Oct. 24, 2024

Related U.S. Application Data
(60) Provisional application No. 63/593,099, filed on Oct. 25, 2023.

(51) Int. Cl. A61B 18/24; A61B 18/00; A61B 18/26

(52) U.S. Cl. CPC A61B 18/245; A61B 18/26; A61B 2018/00059; A61B 2018/00511

(57) ABSTRACT

Devices, systems, and methods for treating stones in anatomical structures are disclosed. A device may include an elongate member and a tubular member extending distally from the elongate member. The elongate member may define one or more lumens with an opening at a distal end of the elongate member. The tubular member may be configured to collapse to a pre-determined shape when in a relaxed state and expand in response to receiving an object. The device may be used with a scope, such as a ureteroscope, lithotripsy devices, and/or other medical devices or systems.

### CROSS REFERENCE TO RELATED APPLICATIONS

The present application claims the benefit of and priority to U.S. Provisional Patent Application Ser. No. 63/593,099, filed on Oct. 25, 2023, the disclosure of which is incorporated herein by reference.

### TECHNICAL FIELD

The present disclosure pertains generally, but not by way of limitation, to medical devices and systems, and methods of treatment. More particularly, the present disclosure relates to medical devices used, for example, in treatments for capturing objects, reducing objects into smaller objects, and removing objects from a subject.

### BACKGROUND

Various devices, systems, and methods have been developed to break kidney stones into smaller fragments and remove kidney stones from patients. These devices and systems are manufactured by any one of a variety of different manufacturing methods and may be used according to any one of a variety of methods for a variety of applications. Of the known medical devices, delivery systems, and methods, each has certain advantages and disadvantages. There is an ongoing need to provide alternative medical devices and delivery devices as well as alternative methods for manufacturing and using medical devices and delivery devices, such as those susceptible to migration in the anatomy.

### BRIEF SUMMARY

This disclosure provides design, material, manufacturing method, and use alternatives for medical devices.

In a first example, a medical device may comprise an elongate member defining one or more lumens with an opening at a distal end of the elongate member and a tubular member extending distally from the distal end of the elongate member, and wherein the tubular member may be configured to collapse to a pre-determined shape when in a relaxed state and expand in response to receiving an object.

Additionally or alternatively to any of the examples above, the tubular member may have a mesh configuration.

Additionally or alternatively to any of the examples above, the mesh configuration may be formed from a braided wire.

Additionally or alternatively to any of the examples above, the tubular member may have a polymer coating covering at least part of the mesh configuration.

Additionally or alternatively to any of the examples above, the tubular member may have a distal end, a containment portion extending distally from the elongate member, and a reduced-diameter portion extending between the containment portion and the distal end.

Additionally or alternatively to any of the examples above, when the tubular member is in the pre-determined shape, an inner diameter of the tubular member may decrease in a proximal direction from the distal end to the reduced-diameter portion and then increases in the proximal direction from the reduced-diameter portion.

Additionally or alternative to any of the examples above, the increase of the inner diameter in the proximal direction from the reduced-diameter portion may be at a rate greater than a rate at which the inner diameter decreases in the proximal direction from the distal end to the reduced-diameter portion.

Additionally or alternatively to any of the examples above, the tubular member may comprise a valve extending across an inner diameter of the tubular member.

Additionally or alternatively to any of the examples above, the valve may be located at a distal end of the tubular member.

Additionally or alternatively to any of the examples above, a distal end of the tubular member may comprise a bumper.

Additionally or alternatively to any of the examples above, the medical device may further include a steerable grasper extending distally from a distal end of the tubular member, the steerable grasper being configured to adjustably grasp the object and position the object within the tubular member.

Additionally or alternatively to any of the examples above, the medical device may further include one or more adjustable members in communication with a distal portion of the tubular member and the one or more adjustable members are configured to be adjusted to cause the tubular member to close around the object.

In a further example, a medical system may comprise an elongate member defining one or more lumens with an opening at a distal end of the elongate member, a tubular member extending distally from the distal end of the elongate member, and a stone treatment device configured to extend along a first lumen of the one or more lumens of the elongate member, and wherein the stone treatment device may be configured to break a stone received in the tubular member into pieces of stone and the tubular member is configured to contain the pieces of stone in the tubular member.

Additionally or alternatively to any of the examples above, the stone treatment device may be a laser lithotripsy device.

Additionally or alternatively to any of the examples above, the tubular member may have a mesh configuration.

Additionally or alternatively to any of the examples above, the tubular member may have a polymer coating covering at least part of the mesh configuration.

Additionally or alternatively to any of the examples above, the tubular member may be configured to collapse to a pre-determined shape when in a relaxed state and expand in response to receiving the stone.

In a further example, a method may comprise delivering a medical device to a renal cavity of a subject, the medical device having an elongate member defining one or more lumens and a tubular member extending distally from a distal end of the elongate member, positioning a stone in the tubular member, applying a stone treatment to the stone in the tubular member to break the stone into pieces, while maintaining the pieces of the stone within the tubular member, and removing the pieces of the stone from the subject.

Additionally or alternatively to any of the examples above, the stone treatment may be a laser lithotripsy treatment.

Additionally or alternatively to any of the examples above, positioning the stone in the tubular member may comprise grasping the stone and moving the stone to a position within the tubular member.

Additionally or alternatively to any of the examples above, maintaining the pieces of the stone within the tubular member may comprise maintaining a valve extending across an inner diameter of the tubular member in a closed position.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative medical device extending into a body of a subject;

FIG. 2 is a schematic side view of an illustrative medical device;

FIG. 3 is a schematic partial cross-section view of the illustrative medical device depicted in FIG. 2;

FIG. 4 is a schematic end view of an illustrative medical device;

FIG. 5 is a schematic side view of an illustrative medical device;

FIGS. 6A-6D depict a schematic flow of an illustrative technique for using a medical device;

FIG. 7 is a schematic side view of an illustrative medical device;

FIGS. 8A and 8B are schematic side views of an illustrative medical device;

FIG. 9 is a schematic side view of an illustrative medical device; and

FIG. 10 is a schematic side view of an illustrative medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Various systems and procedures may be utilized for removing kidney stones from a subject (e.g., a patient). One example procedure is a flexible ureterorenoscopy (fURS) and stone fragmentation procedure. Other procedures for removing kidney stones from a subject are contemplated.

The systems and procedures configured to remove kidney stones from a subject may utilize a scope (e.g., a flexible ureteroscope and/or other suitable scope) inserted through a ureteral sheath that is inserted into a renal cavity of the subject to a location of a stone and may pass through the urethra, the bladder, and the ureter. Once the stone is located, a laser fiber or other suitable lithotripsy device may be inserted through a working channel of the scope and/or the sheath and the stone may be fragmented and/or dusted (e.g., broken into pieces).

When a stone is fragmented, a physician may use a grasper or basket through the scope and/or the sheath to retrieve the stone fragments. The fragments may be removed one by one through extracting and reinserting the scope and/or grasper until as much of the stone is removed from the subject as possible. This repeated process of grasping and removing stone fragments can be time consuming. Further, when performing a fragmentation procedure, care must be taken to ensure the stone fragments are of a desired size for removal from the subject as it may be difficult to remove stone fragments that have a dimension of three millimeters or greater due to an inner diameter of the ureter.

When a stone is dusted, the stone particles may be left in the subject to be naturally flushed out of the kidney and passed over time. When the stone is dusted, a ureteral stent may be left in situ to promote drainage of the stone particles. The implanted stent, however, may be uncomfortable for the patient and a further procedure is required to remove the stent after a period of time (e.g., a week or more). Further, dusting a stone may take more time than fragmenting the stone.

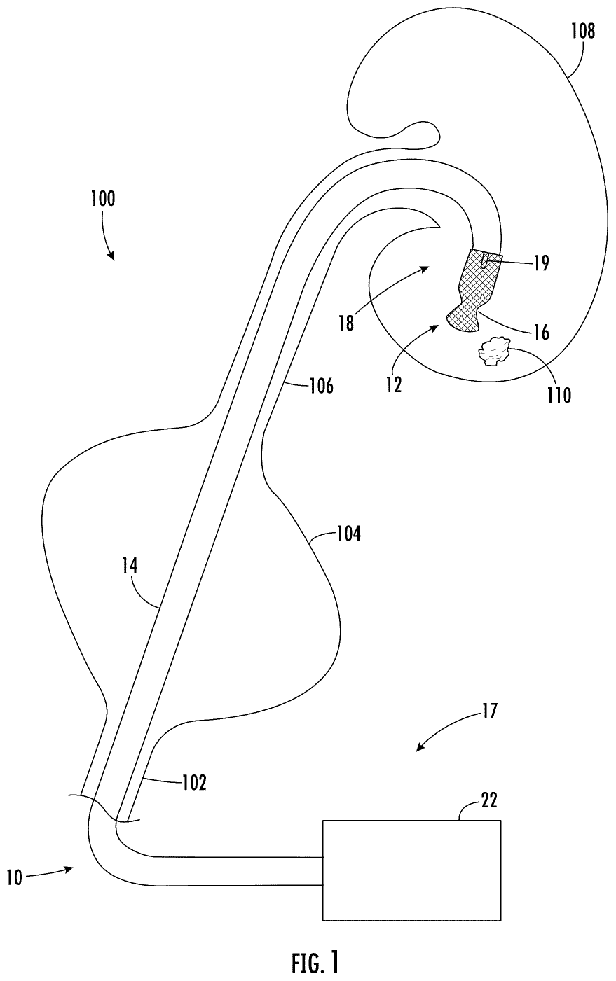

FIG. 1 illustrates a schematic view of a subject's urinary tract 100 with a medical device 10 inserted therein. The urinary tract 100 may include a urethra 102, a bladder 104, a ureter 106, and a kidney 108, among other anatomy. In some instances, a target site may be a stone 110 (e.g., a kidney stone and/or other suitable stone) in the kidney 108 (e.g., in the renal cavity) and/or other location along the urinary tract 100.

In operation, a practitioner (e.g., a physician and/or other medical professional) may insert a distal end 12 of the medical device 10 into the urethra 102, where the medical device 10 may include an elongate member 14 (e.g., an elongate shaft) and a tubular member 16. At a proximal portion 17 of the medical device 10, the elongate member 14 may extend distally from a handle portion 22 and at a distal portion 18 of the medical device 10, the tubular member 16 may extend distally from the elongate member 14.

The practitioner may advance the distal portion 18 of the medical device 10 through the urinary tract 100 such that the distal end 12 at the distal portion 18 of the medical device 10 is positioned proximate a target area (e.g., proximate the stone 110 and/or other suitable target area). In some instances, an imaging device may be utilized to determine a location of stones 110 in the subject.

Although not depicted in FIG. 1, the practitioner may position the tubular member 16 around the stone 110 and/or position the stone within the tubular member 16. In some examples, a practitioner may pass a grasper or grasping device through the elongate member 14 (e.g., via a lumen of a scope and/or a lumen of the elongate member 14) and distal of the tubular member 16 to grasp the stone and draw the stone 110 into the tubular member 16. Alternatively or additionally, in some examples the practitioner may manipulate the tubular member 16 to engage or otherwise position the tubular member 16 around the stone 110.

Once the stone 110 is positioned within the tubular member 16, a dusting or fragmentation procedure, such as a lithotripsy procedure, may be performed on the stone 110. In some examples, a fragmentation or dusting tool 19 may be advanced along the elongate member 14 (e.g., via a lumen of the scope and/or a lumen of the elongate member 14) and into the tubular member 16 and activated to fragment and/or dust the stone 110. In some cases, the fragmentation or dusting tool 19 may be a lithotripsy device, a laser fiber configured to emit a laser beam, an ultrasound device, and/or other suitable devices or systems configured to fragment and/or dust the stone 110.

During and/or after fragmentation and/or dusting, all broken down portions of the stone 110 may be maintained within the tubular member 16 for extraction. The broken-down portions of the stone 110 may be removed by removing the medical device 10 from the subject, by applying a vacuum source through the elongate member 14 and into the tubular member 16, by use of irrigation, and/or in one or more other suitable manners. Such a stone removal device and/or system may reduce procedure times relative to existing fragmentation and dusting techniques, while also achieving removal of all stone debris after the stone removal procedure.

Various additional and/or alternative techniques, devices, or systems may be utilized for breaking down the stone 110 and/or removing the stone from the subject. Example suitable techniques, devices and systems for breaking down and/or removing stones from a subject are disclosed in U.S. Pat. No. 11,382,693, filed on Jan. 31, 2017, and titled MEDICAL DEVICE AND METHODS OF USE, which is hereby incorporated by reference in its entirety for any and all purposes.

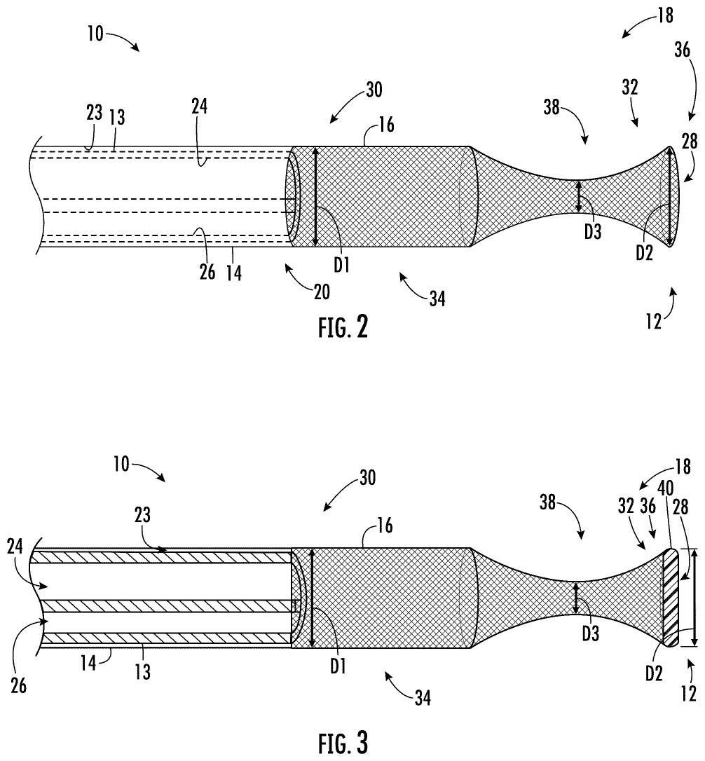

FIG. 2 schematically depicts a side view of a distal portion 18 of an illustrative medical device 10 with a scope 13 (e.g., ureteroscope and/or other suitable scope) inserted through a lumen 23 of the medical device 10. As depicted in FIG. 2, the medical device 10 may include the elongate member 14 having a distal end 20 and the tubular member 16 may extend distally from the distal end 20. The medical device with the scope 13 and/or other suitable stone removal devices or systems may, in combination, be considered a medical system.

The elongate member 14 may be at least partially hollow with a proximal end coupled with the handle portion 22 (FIG. 1) and the distal end 20 coupled with the tubular member 16 such that the tubular member 16 extends distally from the elongate member 14. The elongate member 14 may have any suitable number of lumens. In some examples and as depicted in FIG. 2, the elongate member 14 may have a single lumen 23 with an opening at the distal end 20 of the elongate member 14, but elongate members 14 with other suitable number of lumens are contemplated. The lumen 23 may have any suitable size and/or shape and may be configured to receive a scope (e.g., the scope 13 and/or other suitable scope), receive one or more stone treatment devices, receive one or more control wires, and/or be configured for one or more other suitable purposes.

The elongate member 14 may be any suitable elongate member. For example, the elongate member 14 may be, but is not limited to, a catheter with one or more lumens, a sheath with one or more lumens, a shaft of a scope with one or more lumens, and/or other suitable elongate members. In one example, the elongate member 14 may be a ureteral sheath, but other suitable configurations are contemplated.

The elongate member 14 may have any suitable shape and/or configuration. For example, the elongate member 14 may be circular, ovoidal, irregular, and/or any shape suitable to enter anatomy of a subject. The elongate member 14 may have a uniform shape and/or configuration along its length or a shape and/or configuration that differs along the length of the elongate member 14. In some examples, elongate member 14 may have a varying shape along the length thereof, such as a taper at the distal end 20 to facilitate insertion within the body, but other suitable configurations are contemplated.

Depending upon the particular implementation and intended use, the length of elongate member 14 may vary. Similarly, depending upon the particular implementation and intended use, the elongate member 14 may be rigid along its entire length, flexible along its entire length, or configured for flexure at only certain specified locations. In one example, elongate member 14 may be flexible and adapted for flexible steering within lumens of the anatomy of the subject. For example, the elongate member 14 may include a steering system (not shown) to move at least a portion thereof (e.g., the distal end) and/or a portion of the tubular member 16 up/down and/or side-to-side. Additional degrees of freedom, provided for example via rotation, translational movement of elongate member 14, or additional articulation of bending sections, may also be implemented. Examples of such steering systems may include at least one of pulleys, control wires, gearing, or electrical actuators.

The elongate member 14 may be formed of any suitable material having sufficient flexibility to traverse body cavities and tracts. In some examples, the elongate member 14 may be made of any suitable material that is compatible with living tissue or a living system. That is, the material forming the elongate member may be non-toxic or non-injurious, and it should not cause immunological reaction or rejection. Example suitable materials from which the elongate member 14 may be formed may include, but are not limited to, polymeric materials, metallic materials, polymetric elastomers, rubber, medically approved polyvinylchloride, EVA (Ethylene vinyl acetate), silicone, polyurethane, C-Flex, and/or other suitable materials.

As discussed, the elongate member 14 may receive the scope 13 and/or other suitable devices or systems. The scope 13 may have one or more lumens. For example, as depicted with broken lines in FIG. 2, the scope 13 may include a first lumen 24 and a second lumen 26, but other suitable number of lumens are contemplated. The lumen(s) may be any suitable size and may be configured to provide fluid (e.g., irrigation) to a target area, remove material from a target area via negative pressure, receive a fiber (e.g., a laser fiber, etc.), receive a grasper or grasping device, receive wires (e.g., control wires, guidewires, etc.), receive stone fragments, and/or configured in one or more other suitable manners. The lumen(s) 23 of the elongate member 14 may or may not be configured for similar or different purposes.

The tubular member 16 may extend distally from the distal end 20 of the elongate member 14 and may define an expandable and contracting (e.g., collapsible) lumen 28 that extends from a proximal end 30 to a distal end 32 of the tubular member 16. In some examples, the tubular member 16 and the lumen 28 may be configured to expand as a stone is passed through the lumen 28 and contract and/or collapse around the stone. As such, the tubular member 16 may be biased to its contracted configuration (e.g., its relaxed state), which may be or have a pre-determined shape.

The tubular member 16 may have a first portion 34 (e.g., a containment portion) configured to receive the stone for performing lithotripsy and/or other suitable fragmentation and/or dusting procedure on the stone, a second portion 36 (e.g., a mouth portion) configured to initially receive the stone, and a third portion 38 (e.g., a reduced diameter portion) between the first portion 34 and the second portion 36 or the distal end 32 of the tubular member 16. In some examples, when the tubular member 16 is in a relaxed state (e.g., in its predetermined shape or configuration), the first portion 34 of the tubular member 16 may extend distally from the elongate member 14, the second portion 36 may define at least part of the distal end 32 of the tubular member 16, and the third portion 38 may include a reduced diameter portion of the tubular member 16 that has a smaller inner diameter D3 than an inner diameter DI of the first portion 34 and/or an inner diameter D2 of the second portion 36. For example, an inner diameter of the tubular member 16 may taper in a proximal direction from the distal end 32 to the third portion 38 and then increase in the proximal direction from the third portion 38 to the first portion 34. Although the Figures depict the reduced diameter D3 as being greater than zero, inner surfaces of the third portion 38 of the tubular member 16 may contact one another such that the reduced diameter D3 is zero or there is no inner diameter. The reduced diameter D3 (e.g., a necked-down diameter) of the third portion 38 of the tubular member 16 may be configured to expand to allow stones to pass in a distal-to-proximal direction (e.g., in response to a force pulling a stone into the tubular member 16) and return to the reduced diameter after the stones have passed into the first portion 34 to prevent stones or portions of stones passing in the proximal-to-distal direction.

In some illustrative configurations, one or more of the first portion 34, the second portion 36, and the third portion 38 may have a same diameter as at least one other of the first portion 34, the second portion 36, and the third portion 38 when the tubular member 16 is in a relaxed state. In some examples, the second portion 36 and the third portion 38 may have a same or similar, reduced diameter relative to the first portion 34, which may or may not facilitate inserting the device into body lumens and/or cavities of the anatomy of the subject. In some examples, the first portion, the second portion, and the third portion may have a same diameter when the tubular member 16 is in the relaxed stated, which may be a diameter configured to prevent stones or portions of stones from exiting the tubular member 16 once positioned in the tubular member 16. Other suitable configurations for the pre-determined shape are contemplated.

The proximal end 30 of the tubular member 16 may be coupled with the distal end 20 of the elongate member 14 in any suitable manner. Example suitable manners for coupling the tubular member 16 with the elongate member 14 include, but are not limited to, reflow techniques, welding, heat shrinking, adhesives, and/or other suitable coupling techniques.

The tubular member 16 may have any suitable length. In some examples, the tubular member 16 may have a length suitable to receive the stone in the lumen 28, receive a lithotripsy device via the elongate member 14, and perform lithotripsy procedure on the stone.

The tubular member 16 may be formed in any suitable manner configured to self-contract after expanding to accommodate an object in the lumen 28 and/or accommodate medical components (e.g., a grasper, a treatment device, etc.). For example, the tubular member 16 may be formed as a resilient sheath, a laser cut tube, a knitted tube, a mesh tube, a braided tube, a wound tube, a woven tube, and/or may be formed in one or more other suitable manners. In one example, the tubular member 16 may have a mesh configuration formed with a braided wire coupled with the elongate member 14. In some cases, the tubular member 16 may have a stent configuration.

The tubular member 16 may be formed from one or more suitable materials. Example suitable materials include, but are not limited to, polymeric materials, metallic materials, fabric materials, metal alloys, shape memory alloys, polymers, nickel-titanium alloys, cobalt-chromium-nickel-molybdenum alloys, and/or other suitable materials enabling the tubular member 16 to be expanded into a shape when positioned at a target site. In some instances, the material may be selected to enable the tubular member 16 to be inserted into and/or removed from the anatomy of the subject with relative case. In some examples, the tubular member 16 may be formed from alloys such as, but not limited to, nitinol and/or Elgiloy®. In one example, the tubular member 16 may be braided using one or more wires formed from nitinol.

FIG. 3 depicts a partial cross-sectioned view and partial side view of an illustrative configuration of the distal portion 18 of the medical device 10, wherein the elongate member 14 and the scope 13 are depicted in cross-section and a side view of the tubular member 16 is depicted. In some configurations, the distal end 32 of the tubular member 16 may include a bumper 40 (e.g., a soft tip). The bumper 40 may define a terminal end of the medical device 10 and may be configured to facilitate insertion of the medical device 10 into and/or through the anatomy of the subject to the target area by mitigating or reducing traumatic contact with the anatomy relative to when the bumper 40 is not utilized.

The bumper 40 may be configured in any suitable manner to facilitate insertion of the tubular member 16 into the anatomy of the subject. In some examples, the bumper 40 may have a tapered configuration with an outer diameter that reduces in a proximal-to-distal direction, rounded edges or surfaces, and/or other suitable configurations designed to facilitate insertion of the tubular member 16 to the target site.

The bumper 40 may be formed from any suitable material configured to be atraumatic as the tubular member 16 passes through the anatomy of the subject. Example suitable materials for the bumper 40 include, but are not limited to metallic materials, polymeric materials, soft materials, resilient materials, silicone, hydrogels, collagen, elastin, polyurethane, polyethylene, polytetrafluoroethylene (PTFE), polydimethylsiloxane (PDMS), polyglycolic acid (PGA), polylactic acid (PLA), gelatin, chitosan, alginate, polyvinylpyrrolidone (PVP), polycaprolactone (PCL), natural rubber, polyethylene glycol (PEG), and/or other suitable materials. In some examples, the bumper 40 may be formed from silicone and/or other suitable materials.

In some examples, the material(s) used to form the bumper 40 may be of a low durometer. Example suitable Shore durometer hardness readings for the material of the bumper 40 may include, but are not limited to, 60 A or less, 50 A or less, 40 A or less, 30 A or less, 20 A or less, and/or other suitable values.

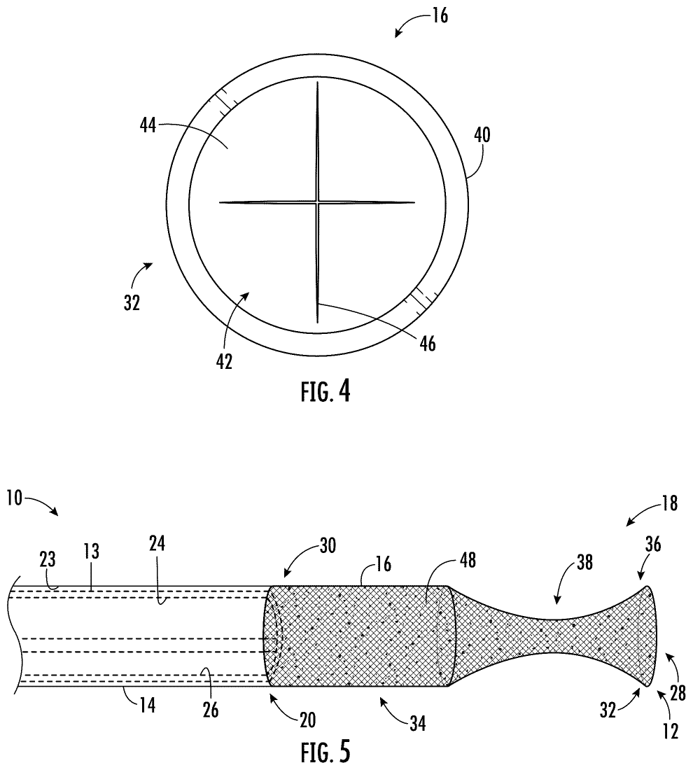

FIG. 4 depicts a schematic distal end view of the distal portion 18 of the illustrative medical device 10 depicted in FIG. 3. In some cases, the bumper 40 may extend across an opening 42 at the distal end 32 of the tubular member 16 and form a valve 44 extending across a diameter (e.g., an inner diameter) of the tubular member 16. Alternatively, the valve 44 may be formed separate from the bumper 40. The valve 44 may have a slit 46 (e.g., a cross-slit, as depicted in FIG. 4, and/or other suitable slits) and/or other suitable opening configured to be biased closed and selectively opened in response to engagement with a tool (e.g., a grasper, etc.) and/or a stone coupled with the tool.

Although the valve 44 is depicted as being at the opening 42 (e.g., at the distal end 32) of the tubular member 16, the valve 44 may be located at other suitable locations along a length of the tubular member 16 distal of a location at which a procedure (e.g., a lithotripsy procedure) is to be applied to a stone received within the tubular member 16. In some examples, the valve 44 may be located at an axial location along the length of the tubular member 16 having a greatest diameter to limit an amount of stretch or expansion that is required by the valve 44 to maintain the valve 44 in a closed position when tubular member 16 expands. In other examples, the valve 44 may be configured to expand with the tubular member 16 and may be located at a smallest diameter axial location (e.g., a necked-down portion, such as the third portion 38 of the tubular member 16). When the valve 44 is located at the smallest diameter axial location, the tubular member 16 at the smallest diameter axial location may return to a relaxed, contracted state after a stone pass therethrough and before a procedure is performed on the stone such that the valve 44 is in a closed position when the procedure is performed.

In operation, the tool may be inserted distally through the slit 46 or other opening of the valve 44, engage a stone, and withdraw the stone through the slit 46, and withdraw the stone from the subject and/or perform a procedure on the stone, as discussed herein or otherwise, while maintaining the valve 44 in the closed configuration. In some examples, the valve 44 may automatically close without an opening force sufficient to open the valve 44 being applied thereto. As the stone is broken into separate pieces, the separate pieces may remain within the tubular member 16 and may be prevented from exiting the opening 42 by the valve 44 remaining in a closed or blocking configuration.

FIG. 5 depicts a schematic side view of an illustrative configuration of the distal portion 18 of the medical device 10, with a coating 48 (e.g., an internal covering, an external covering, and/or other suitable coating). When included, the coating 48 may be applied to and/or cover at least part of or an entire circumference of the tubular member 16. When the tubular member 16 is formed in a porous configuration (e.g., with woven or braided wires, with a cut tube, etc.), the coating 48 may be configured to prevent stone particles (e.g., fragments, dust, etc.) from escaping through pores and/or other openings in the tubular member 16. In some examples, the coating 48 may be watertight around the tubular member 16.

The coating 48 may be formed from any suitable material configured to cover pores or openings along the tubular member 16 and/or configured for other purposes. In some examples, the coating 48 may be made from a material selected or configured to mitigate or prevent trauma or injury to tissue of the subject as the tubular member 16 is delivered to a target area. Example suitable materials for the coating 48 include, but are not limited to metallic materials, polymeric materials, fabric materials, soft materials, resilient materials, silicone, hydrogels, collagen, elastin, polyurethane, polyethylene, polytetrafluoroethylene (PTFE), polydimethylsiloxane (PDMS), polyglycolic acid (PGA), polylactic acid (PLA), gelatin, chitosan, alginate, polyvinylpyrrolidone (PVP), polycaprolactone (PCL), natural rubber, polyethylene glycol (PEG), and/or other suitable materials. In some examples, the coating 48 may be formed from silicone and/or other suitable materials. In some examples, the material(s) used to form the coating 48 may be of a low durometer. Example suitable Shore durometer hardness readings for the material of the coating 48 may include, but are not limited to, 60 A or less, 50 A or less, 40 A or less, 30 A or less, 20 A or less, and/or other suitable values. Further, although not required, the coating 48 may be configured to increase an elasticity of the tubular member 16 when compared to an elasticity of a tubular member 16 without the coating 48.

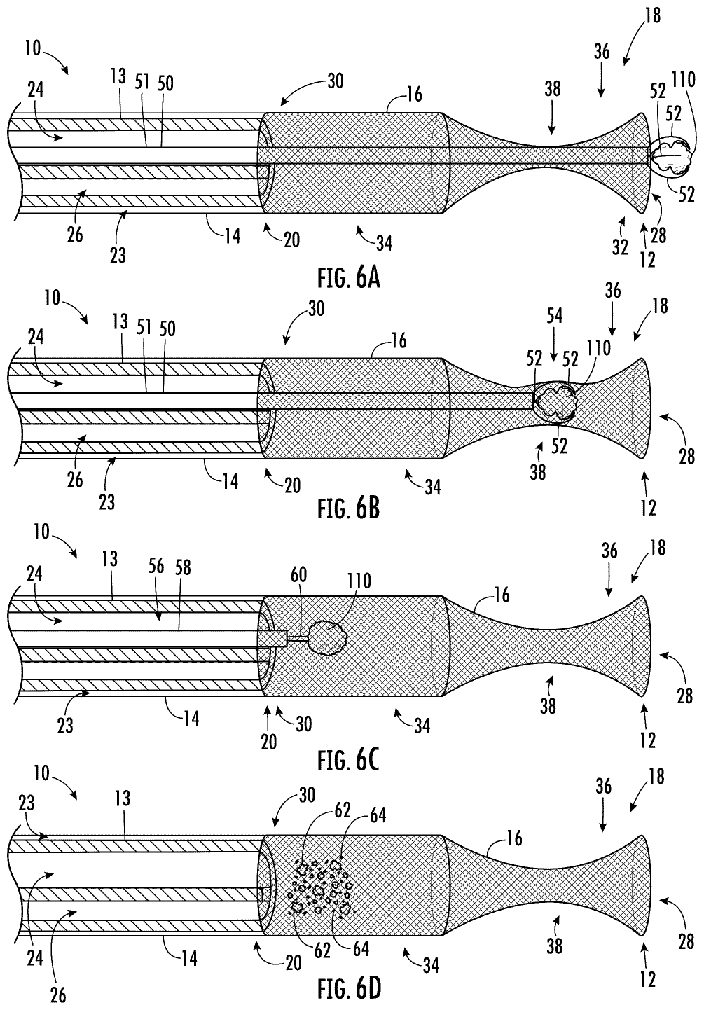

FIGS. 6A-6D depict a schematic flow of a technique for capturing a stone 110 and applying a procedure (e.g., a lithotripsy procedure and/or other suitable procedure) to the stone 110. The medical device 10 is schematically depicted in FIGS. 6A-6D with the elongate member 14 in cross-section and the tubular member 16 in a side view.

As depicted in FIG. 6A, a grasper 50 (e.g., a steerable grasper) or other suitable stone-engaging device may be inserted through a lumen of the scope 13 to a location distal of the distal end 12 of the medical device 10 and adjustably grasp the stone 110. In some cases, the grasper 50 or other suitable device may be inserted through the first lumen 24 of the scope 13, as depicted in FIG. 6A, and/or other suitable working lumen including, but not limited to the lumen 23 of the elongate member 14 independent of the scope 13.

Any suitable grasper 50 or other suitable device configured to engage the stone 110 and position the stone 110 within the tubular member 16 may be utilized. In some examples, the grasper 50 may include one or more paddles or prongs 52 extending distally from an elongate shaft 51 (e.g., a tube and/or other suitable shaft structure) and configured to selectively grasp the stone 110. In some examples, the grasper 50 may include one or more baskets in addition to or as an alternative to the paddles or prongs 52.

The grasper 50 be controlled by a user (e.g., manually) or a robot (e.g., electronically, which may or may not be directed by a user) at or proximate a proximal end of the medical device 10. In some examples, the paddles or prongs 52 may be selectively actuatable to receive the stone 110 between the paddles or prongs 52. Additionally or alternatively, a distal end of the grasper 50 and/or the paddles or prongs 52 may be directionally controlled (e.g., steered) using pull wires and/or other control mechanisms.

Once the stone 110 is grasped, the grasper 50 may be drawn into the tubular member 16, as depicted in FIG. 6A. When the stone 110 and/or the grasper 50 has a diameter or widest point passing through the tubular member 16 that is greater than an inner diameter of the tubular member 16, the tubular member 16 may expand to accommodate the stone 110 and/or the grasper 50 at one or more expansion locations 54 (e.g., at the third portion 38, as depicted in FIG. 6A, but other locations are contemplated). Once the stone 110 and/or the grasper 50 passes an axial location of the tubular member 16 with an inner diameter smaller than the diameter or widest portion of the stone 110 and/or the grasper 50, the tubular member 16 may be biased to return to and automatically return to a relaxed, contracted configuration. Further, although not depicted, the stone 110 may be drawn into the first portion 34 of the tubular member 16, released from the grasper 50 and/or other suitable device, and the grasper 50 and/or other suitable device may be withdrawn from the first lumen 24 of the scope 13 and the medical device 10. Alternatively or additionally, the grasper 50 and/or other suitable device may remain in the first lumen 24 while a procedure is performed on the stone 110.

Once the stone 110 is positioned at a desired location within the tubular member 16 (e.g., in the first portion 34 of the tubular member 16), a stone treatment device 56 (e.g., a lithotripsy device and/or other suitable device configured to fragment or dust the stone 110, such as the fragmentation and/or dusting tool 19) may be delivered to the tubular member 16 through the first lumen 24, as depicted in FIG. 6C, or other suitable lumen including, but not limited to the lumen 23 of the medical device 10 independent of the scope 13. In some cases, the stone treatment device 56 may be a laser lithotripsy device including a laser fiber 58 configured to emit a laser 60, but other suitable stone treatment devices 56 and/or other suitable stone fragmenting or dusting devices are contemplated.

The stone treatment device 56 and/or other suitable device may be positioned adjacent the stone and the laser 60 or other suitable medium may be applied to the stone 110 to break the stone into pieces (e.g., dust and/or particles). In some cases, the scope 13 may include one or more imaging components that may facilitate observing the stone 110 and/or the application of the laser 60 or other suitable lithotripsy treatment to the stone 110 and facilitate how the laser 60 may need to be manipulated to continually break up the stone 110 within the tubular member 16.

Once the stone 110 has been broken into fragments 62 and/or dust 64 (e.g., pieces of stone), as depicted for example in FIG. 6D, the stone treatment device 56 and/or other suitable device may be removed from the first lumen 24. Then, the medical device 10 with the fragments 62 and/or dust 64 secured within the tubular member 16 may be withdrawn from the subject without leaving fragments or dust within the subject. Alternatively or additionally, the lithotripsy device and/or other suitable device may be withdrawn from the subject with the medical device 10 and the fragments 62 and/or dust 64 within the tubular member 16.

In some examples, the second lumen 26 and/or other suitable lumen of the scope 13 or a lumen of the medical device 10 may be an aspiration lumen. When an aspiration lumen is included, a negative pressure may be applied to second lumen 26 or other suitable lumen to draw the fragments 62 and/or the dust 64 into and through the second lumen 26 or other suitable lumen to remove the fragments 62 and/or the dust 64 from the tubular member 16 and the subject. In some cases, the negative pressure may be applied to the second lumen 26 and/or other suitable lumen during fragmentation and/or dusting of the stone (e.g., during the lithotripsy procedure and/or other suitable procedure).

In addition to utilizing negative pressure to remove stone fragments 62 and/or dust 64 from the tubular member 16, irrigation may be applied to the lumen 28 of the tubular member 16 through one or more lumens of the scope 13 and/or the elongate member 14. When included, the irrigation may be applied to the lumen 28 and aspirated out of the lumen 28 with fragments 62 and/or dust 64 through an aspiration lumen.

One or more additional and/or alternative steps or features may be included in the process of removing a stone from a subject relative to what is depicted in FIGS. 6A-6D. Utilizing the medical device 10 as discussed may facilitate reducing procedure time, increase stone clearance rate, reduce a need for post-operative stenting, and reduce a risk for relapse caused by unretrieved stones or stone particles. Further, although FIGS. 6A-6D depict the structure of the tubular member 16 with wide pores or openings, these are not necessarily drawn to scale and any pores and/or openings along the length of the tubular member 16 may be configured to prevent or mitigate unintentional release of stone fragments or dust.

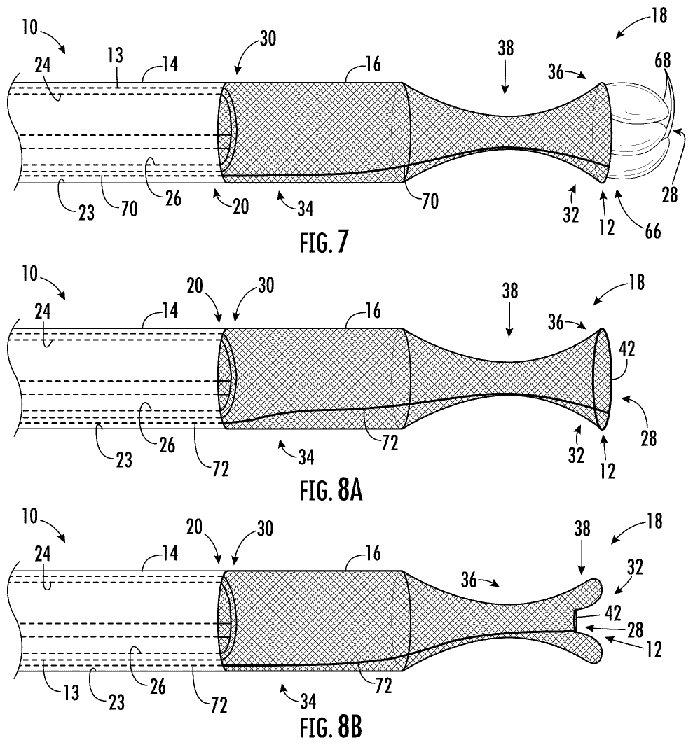

FIG. 7 depicts a schematic side view of the distal portion 18 of an illustrative configuration of the distal end 18 of the medical device 10, with a grasping system 66 (e.g., a steerable grasping system or grasper) positioned at or at least partially positioned at the distal end 32 of the tubular member 16. Although the grasping system 66 is depicted in FIG. 7 as being at least partially positioned at the distal end 32 of the tubular member 16, the grasping system 66 may be located at other suitable locations along the length of the tubular member 16.

The grasping system 66 may take on any suitable configuration for engaging stones in a subject and drawing the stones into tubular member 16. When included, the grasping system 66 may eliminate a need for using a separate stone grasping device (e.g., the grasper 50 and/or other suitable stone engaging device). In some examples, the grasping system 66 may include one or more paddles 68 coupled with one or more control wires 70 (e.g., one or more adjustable members) and the distal end of the tubular member 16. In one example, the grasping system 66 may include three paddles 68 and a control wire 70 coupled to the paddles to manipulate the paddles 68 between opened and closed positions. The control wire 70 may extend from the paddles 68 through one or more lumens of the elongate member 14 (e.g., the lumen 23, as depicted in FIG. 7) and/or one or more lumens of the scope 13.

In operation, a user or robot may manipulate the control wire(s) 70 at a proximal end of the medical device 10 to adjust the paddles 68 to a closed position for delivery to the target area in the subject. In some cases, the paddles 68 may have a configuration (e.g., a distally tapering configuration) when in the closed position that facilitates delivery of the medical device to the target area. Further, the paddles 68 may be formed from a soft and/or resilient material to prevent or mitigate trauma to tissue of the subject during delivery and/or to facilitate engaging the stone at the target area. Once at the target area proximate a stone, the control wire(s) 70 may be manipulated to open the paddles 68 and the tubular member 16 may be adjusted distally such that the stone is positioned adjacent to and/or within the paddles 68. Alternatively or additionally, the paddles may be biased on the opened position such that the paddles 68 may automatically adjust to the opened position when a tension on the control wire(s) 70 is released. The control wire(s) 70 may be manipulated to close the paddles 68 around the stone and draw the stone to a desired location in the tubular member 16 for performing a lithotripsy procedure and/or other procedure on the stone. Other suitable steps or features of utilizing the medical device 10 with paddles 68 and/or other suitable grasping components attached to the distal end 32 of the tubular member 16 are contemplated.

FIGS. 8A and 8B depict a schematic side view of an illustrative configuration of the distal portion 18 of the device 10, with a control wire 72 (e.g., an adjustable member) extending through the medical device 10 and engaging the distal end 32 of the tubular member 16. The control wire 72 may be manipulated to cause the distal end 32 of the tubular member 16 to close and to be drawn into the lumen 28. Although only a single control wire 72 is depicted in FIGS. 8A and 8B, multiple control wires 72 may be utilized, as desired.

As depicted in FIG. 8A, the control wire(s) 72 may extend through the medical device 10 in one or more lumens of the elongate member 14 (e.g., the 13, as depicted in FIGS. 8A and 8B) and/or one or more lumens of the scope 13, extend in the lumen 28 of the tubular member 16, and couple with the distal end 32 of the tubular member 16. The control wires(s) 72 may engage the distal end 32 of the tubular member 16, such that a tension may be applied to the control wire(s) 72 to close or at least reduce a diameter of the opening 42 at the distal end 32 of the tubular member 16 and draw the distal end 32 into the lumen 28 of the tubular member, as depicted for example in FIG. 8B. Further, in some cases, the control wire(s) 72 may be utilized to control a direction of movement of the tubular member 16 within the subject.

In operation, a user or robot may position the distal end 32 of the tubular member 16 (e.g., via manipulating the control wire(s) 72 and/or via other suitable control mechanisms) at the target area within the subject. The distal end 32 of the tubular member 16 may be in the opened position or a closed position during delivery, as desired. Further, as discussed above, the tubular member 16 may include a coating 48 or bumper 40 formed from a soft and/or resilient material to prevent or mitigate trauma to tissue of the subject during delivery and/or to facilitate engaging the stone at the target area.

Once at the target area proximate a stone, the control wire(s) 72 may be manipulated to position the distal end 32 of the tubular member 16 at and/or around the stone such that the stone is positioned adjacent to and/or within the opening 42 at the distal end 32 of the tubular member 16. The control wire(s) 72 may be manipulated to close the opening 42 with the stone in the lumen 28 of the tubular member 16 and the control wire(s) 72 may be further manipulated to draw the distal end 32 of the tubular member 16 proximally to position the stone at an axial location with the tubular member 16 sufficient for performing a lithotripsy procedure and/or other suitable procedure on the stone. The distal end 32 of the tubular member 16 may remain in a closed and/or withdrawn position during the procedure and/or during removal from the medical device 10, as desired.

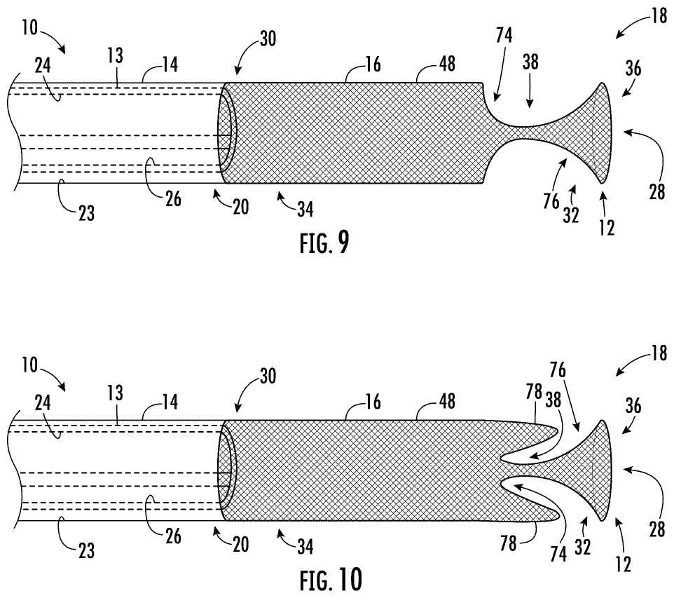

The tubular member 16 may have any suitable configuration, as discussed herein or otherwise. In some examples, the tubular member 16 may have a shape and/or size configured to facilitate receiving a stone or other object and preventing the stone or other object, or a portion thereof, from exiting through an opening at the distal end 32 of the tubular member 16. FIGS. 9 and 10, discussed below, depict illustrative configurations of the tubular member 16 that are configured to facilitate receiving stones and preventing stones or portions thereof from exiting the opening at the distal end 32 of the tubular member 16.

FIG. 9 depicts a schematic side view of an illustrative configuration of the distal portion 18 of the medical device 10, where a first transition 74 (e.g., a proximal curve) between a distal end of the first portion 34 of the tubular member 16 and the third portion 38 of the tubular member 16 is sharper than or tapers in a distal direction at a greater rate than a taper in a proximal direction of a second transition 76 (e.g., a distal curve) between the second portion 36 of the tubular member 16 and the third portion 38. The tapers of the first transition 74 and the second transition 76 may be at any suitable rate of taper in the distal direction and the proximal direction, respectively, as desired. In some examples, the second transition 76 may be a gradual transition or taper and the gradual transition or taper between the second portion 36 and the third portion 38 of the tubular member 16 may facilitate receiving a stone in the first portion 34 of the tubular member 16. Additionally or alternatively, the first transition 74 may be a steep transition or taper (e.g., relative to the gradual transition of the second transition 76) and the steep transition or taper may frustrate and/or prevent the stone or pieces thereof from exiting the first portion 34 of the tubular member 16.

FIG. 10 depicts a schematic side view of an illustrative configuration of the distal portion 18 of the medical device 10, where the tubular member 16 includes one or more lobes 78, the first transition 74 (e.g., a proximal curve) between a distal end of the first portion 34 of the tubular member 16 and the third portion 38 of the tubular member 16, and the second transition 76 (e.g., a distal curve) between the second portion 36 of the tubular member 16 and the third portion 38. In some examples and similar to as discussed above with respect to FIG. 9, the first transition 74 may be sharper than the second transition 76 such that the second transition 76 facilitates receiving the stone into the first portion 34 of the tubular member 16 and the first transition 74 prevents or mitigates the likelihood that portions of the stone will enter the third portion 38 and exit through a distal end of the tubular member 16.

The lobes 78 may be configured to receive or capture fragments or portions of a stone received in the lumen 28 of the tubular member 16 after the stone has been fragmented and/or dusted. To facilitate creating volume to receive the fragmented and/or dusted stone and preventing the fragments and/or dust from exiting the tubular member 16 via the third portion 38, the lobes 78 may extend distally over the first transition 74, the third portion 38, the second transition 76, and/or over one or more other suitable portions of the tubular member 16. In some examples, the distal extension of the lobes 78 may facilitate creating a sharp transition between the first portion 34 and the third portion 38 of the tubular member 16 and mitigate a likelihood that portions of the stone will enter the third portion 38.

The tubular member 16 may include one or more lobes 78. In some examples, the tubular member 16 may include a single lobe 78 extending circumferentially around or at least partially around a circumference of the tubular member 16. Alternatively, in some examples, the tubular member 16 may include two or more separate lobes 78 spaced from one another (e.g., circumferentially spaced and/or spaced in one or more other suitable manners).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device, comprising:
an elongate member defining one or more lumens with an opening at a distal end of the elongate member; and
a tubular member extending distally from the distal end of the elongate member, and
wherein the tubular member is configured to collapse to a pre-determined shape when in a relaxed state and expand in response to receiving an object.

2. The medical device of claim 1, wherein the tubular member has a mesh configuration.

3. The medical device of claim 2, wherein the mesh configuration is formed from a braided wire.

4. The medical device of claim 2, wherein the tubular member has a polymer coating covering at least part of the mesh configuration.

5. The medical device of claim 1, wherein the tubular member has a distal end, a containment portion extending distally from the elongate member, and a reduced-diameter portion extending between the containment portion and the distal end.

6. The medical device of claim 5, wherein when the tubular member is in the pre-determined shape, an inner diameter of the tubular member decreases in a proximal direction from the distal end to the reduced-diameter portion and then increases in the proximal direction from the reduced-diameter portion.

7. The medical device of claim 6, wherein the increase of the inner diameter in the proximal direction from the reduced-diameter portion is at a rate greater than a rate at which the inner diameter decreases in the proximal direction from the distal end to the reduced-diameter portion.

8. The medical device of claim 1, wherein the tubular member comprises a valve extending across an inner diameter of the tubular member.

9. The medical device of claim 1, wherein a distal end of the tubular member comprises a bumper.

10. The medical device of claim 1, further comprising:
a steerable grasper extending distally from a distal end of the tubular member, the steerable grasper being configured to adjustably grasp the object and position the object within the tubular member.

11. The medical device of claim 1, further comprising:
one or more adjustable members in communication with a distal portion of the tubular member and the one or more adjustable members are configured to be adjusted to cause the tubular member to close around the object.

12. A medical system, comprising:
an elongate member defining one or more lumens with an opening at a distal end of the elongate member;
a tubular member extending distally from the distal end of the elongate member; and
a stone treatment device configured to extend along a first lumen of the one or more lumens of the elongate member, and
wherein the stone treatment device is configured to break a stone received in the tubular member into pieces of stone and the tubular member is configured to contain the pieces of stone in the tubular member.

13. The medical system of claim 12, wherein the stone treatment device is a laser lithotripsy device.

14. The medical system of claim 12, wherein the tubular member has a mesh configuration.

15. The medical system of claim 14, wherein the tubular member has a polymer coating covering at least part of the mesh configuration.

16. The medical system of claim 12, wherein the tubular member is configured to collapse to a pre-determined shape when in a relaxed state and expand in response to receiving the stone.

17. A method, comprising:
delivering a medical device to a renal cavity of a subject, the medical device having an elongate member defining one or more lumens and a tubular member extending distally from a distal end of the elongate member;
positioning a stone in the tubular member;
applying a stone treatment to the stone in the tubular member to break the stone into pieces, while maintaining the pieces of the stone within the tubular member; and
removing the pieces of the stone from the subject.

18. The method of claim 17, wherein the stone treatment is a laser lithotripsy treatment.

19. The method of claim 17, wherein positioning the stone in the tubular member comprises grasping the stone and moving the stone to a position within the tubular member.

20. The method of claim 17, wherein maintaining the pieces of the stone within the tubular member comprises maintaining a valve extending across an inner diameter of the tubular member in a closed position.
